(19) United States
(12) Patent Application Publication
(10) Pub. No.: US 2020/0000653 A1
(43) Pub. Date: Jan. 2, 2020

(54) DEVICE, SYSTEM AND METHOD FOR MEDICAL EVACUATION

(71) Applicant: AEROMEDICAL GROUP LTD, Rehovot (IL)

(72) Inventors: Michael OREN, Rehovot (IL); Gary GROSSMAN, Rehovot (IL); Ariel BRAVERMAN, Rishon LeTzion (IL); Roni GUREVICH, Petach Tikva (IL)

(73) Assignee: AEROMEDICAL GROUP LTD, Rehovot (IL)

(21) Appl. No.: 16/465,175

(22) PCT Filed: Nov. 28, 2017
(86) PCT No.: PCT/IL2017/051290

§ 371 Date: May 30, 2019

(30) Foreign Application Priority Data
Nov. 30, 2016 (IL) 249302

(51) Int. Cl. A61G 1/013; A61B 5/0205; A61G 1/04

(52) U.S. Cl. CPC A61G 1/013; A61B 5/02055; A61H 31/008; A61B 2505/01; A61B 2562/225; A61G 1/04

(57) ABSTRACT

Embodiments of the invention are related to a foldable stretcher that includes, a foldable fabric, one or more sensors for detecting a medical condition of a patient, attached to the foldable fabric and configured to connect to one or more external medical condition monitoring units; and one or more treatment supplying channels for supplying medical treatment to the patient and configured to connect to one or more treatment supplying units.

### BACKGROUND OF THE INVENTION

Emergency evacuation of casualties requires fast methods of medical evaluation and treatment of patients. A patient is usually given an initial medical evaluation and initial treatment by a paramedic on the field. The paramedic also prepares the patient for further treatment in an evacuation vehicle such as an ambulance or an air-ambulance. For example, intravenous (IV) infusion or a preparation for giving IV infusion may be performed on the field by inserting a cannula to the patient. In yet another example, a mask for artificial ventilation, (also known as artificial respiration) may be placed on the patient's face. The paramedic may further connect the patient to several diagnostic tools, such as a blood pressure sleeve, a pulse oximetry device, electrocardiography (ECG) electrodes, and the like.

Stretchers for evacuation of patients are either foldable or rigid portable beds for out-of-hospital care situations. There are several widely used types of stretchers, litters, scoop stretchers, ambulance stretchers and wheeled stretchers. All this stretchers include beds/blankets for supporting the patients and means to carry the patients.

Accordingly, a patient being giving a first aid/first medical diagnosis on the field when carried out to an ambulance (air ambulance, evacuation helicopter, etc.) will have to be moved from a litter to the ambulance stretcher and disconnected from the diagnostic tools, of the paramedic on the field, and optionally also from the infusion and reconnect tool by tool to the ambulance's diagnostic/treatment equipment. These operations can take minutes which may be critical to the patient's conditions.

### SUMMARY

Some embodiments of the invention are related to a foldable stretcher that includes, a foldable fabric, one or more sensors for detecting a medical condition of a patient, attached to the foldable fabric, one or more treatment supplying channels for supplying medical treatment to the patient, attached to the foldable fabric and a plug configured to connect the one or more sensors and the one or more treatment supplying channels to an external medical condition monitoring unit and an external medical treatment supplying unit in a single plugging act.

Some embodiments of the invention are related to a foldable stretcher that includes, a foldable fabric, one or more sensors for detecting a medical condition of a patient, attached to the foldable fabric and configured to connect to one or more external medical condition monitoring units; and one or more treatment supplying channels for supplying medical treatment to the patient and configured to connect to one or more treatment supplying units.

Some embodiments of the invention are related to a plug for connecting treatment supplying channels and life supporting sensors of a stretcher, to an external medical condition monitoring unit and an external medical treatment supplying unit. The plug may include, a housing connectable to a contra plug, a first portion configured to connect one or more sterile treatment supplying channels, wherein the first portion is sterile, and a second portion configured to connect at least one of: a wire and a channels.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is an illustration of a foldable stretcher according to some embodiments of the invention;

FIG. 1B is a high level block diagram of a controller according to some embodiments of the invention; and

FIGS. 2A and 2B are illustrations of a plug according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

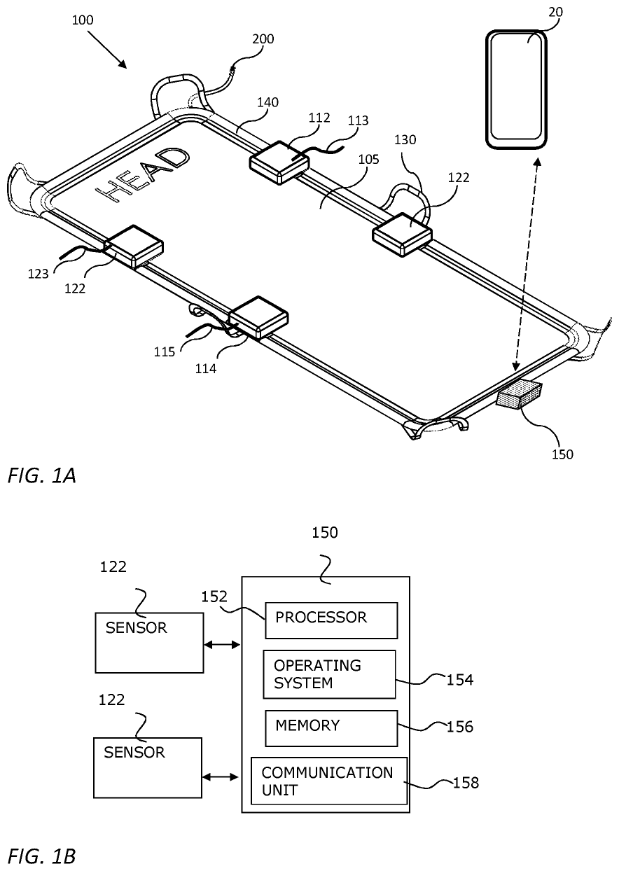

Some aspects of the invention are related to foldable stretchers for rapid evacuation of wounded patients from car accidents, war fields, natural disasters and the like. Reference is now made to FIG. 1A which is an illustration of a foldable stretcher 100 according to some embodiments of the invention. Foldable stretcher 100 may include a foldable fabric 105, one or more treatment supplying channels 112 and 114 for supplying medical treatment to the patient and one or more sensors 122 and 124 for detecting a medical condition of a patient. In some embodiments, foldable stretcher 100 may further include a plug 200 and/or a controller 150. Foldable stretcher 100 may further include handles 130 for carrying foldable stretcher 100 and optionally a foldable stretcher frame 140. In some embodiments, treatment supplying channels 112 and 114 and/or one or more sensors 122 and 124 are attached or integrated into foldable fabric 105.

Foldable fabric 105 may be any suitable fabric known in the art for making stretchers. One or more treatment supplying channels 112 may include a liquid line (e.g., tube) and a sterile connector for connecting a cannula for IV infusion and treatment supplying channels 114 may include pressurized air/oxygen supply tube and a connector for connecting, for example, a respiration mask. Treatment supplying channels 112 may be sealed and kept sterile.

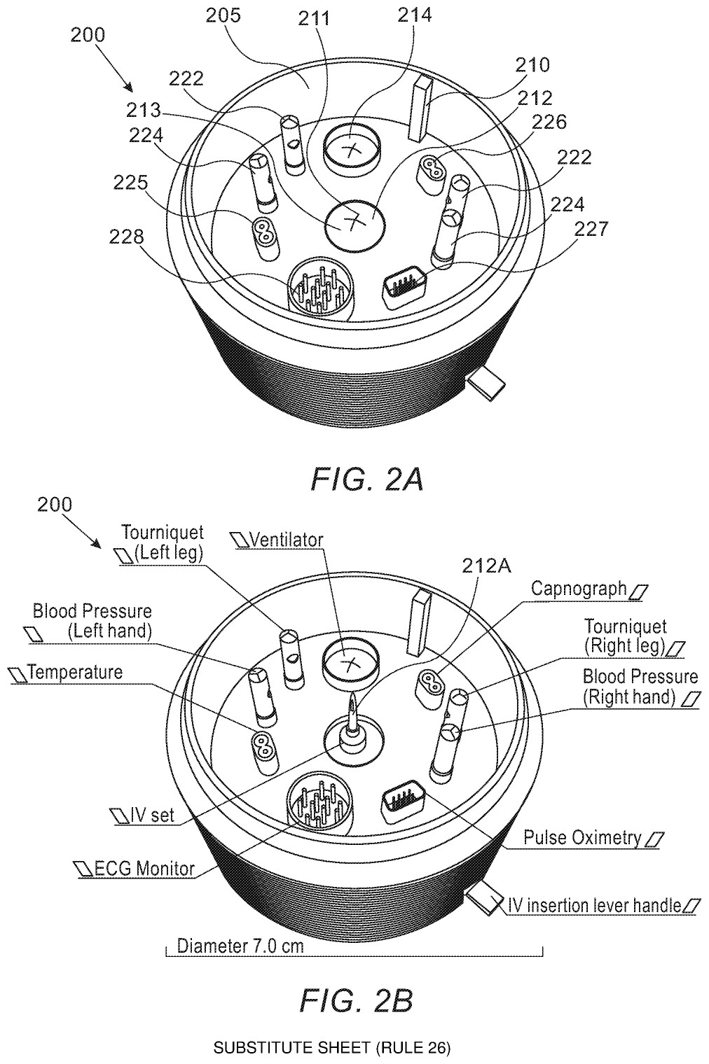

In some embodiments, one or more sensors 122 may be configured to connect to one or more external medical condition monitoring units. In some embodiments, one or more treatment supplying channels 112 and 114 may be configured to connect to one or more treatment supplying units. In some embodiments, treatment supplying channels 112 and 114 may be connected to plug 200, for example, using a sterile connection, as illustrated and explained with respect to FIGS. 2A and 2B. In some embodiments, at least portions of the tubes connecting the one or more treatment supplying channels 114 and 112 are embedded into the fabric of fabric stretcher 105 or the foldable stretcher frame 140.

In some embodiments, one or more sensors 122 and 124 may be connected to plug 200 using any suitable wires. The wires may be attached or integrated into foldable stretcher 100. One or more sensors 122 and 124 may include blood pressure sleeve, a pulse oximetry device, electrocardiography (ECG) electrodes, thermometer, capnography sensor, and the like. In some embodiments, one or more sensors 122 may be configured to send information (e.g., measurements of medical parameters) to controller 150.

In some embodiments, plug 200 is configured to connect one or more sensors 122 and 124 and the one or more treatment supplying channels 114 and 112 to an external medical condition monitoring unit and an external medical treatment supplying unit in a single plugging act as illustrated and discussed with respect to FIGS. 2A and 2B.

Additionally or alternatively, each of one or more sensors 122 may further include a connector 123 for connecting the sensor to one or more external medical condition monitoring units. In some embodiments, each one of one or more treatment supply channels 112 and 114 may further include a connector 113 and 115 for connecting the channel to a medical supply unit. Connectors 113 and/or 115 may be sterile or not.

Reference is now made to FIG. 1B with is a high level block diagram of controller 150 according to some embodiments of the invention. Controller 150 may include a processor 152 that may be, for example, a chip or any suitable computing or computational device, an operating system 154 and a memory 156. Processor 152 may be configured to carry out methods according to embodiments of the present invention by for example executing instructions stored in a memory such as memory 156. Controller 150 may further include a communication unit 158, for example a wireless communication unit (e.g., Bluetooth module) and/or wired communication unit, such as, a USB connector.

Operating system 154 may be or may include any code segment designed and/or configured to perform tasks involving coordination, scheduling, arbitration, supervising, controlling or otherwise managing operation of controller 150, for example, scheduling execution of programs. Memory 156 may be or may include any suitable memory units or storage units. Memory 156 may be or may include a plurality of, possibly different memory units.

In some embodiments, controller 150 may be configured to receive from one or more sensors 122 a medical information, for example, blood pressure measurements, heartrate measurements, and the like. In some embodiments, controller 150 may be configured to store the medical information in memory 156. The information in memory 156 may be accessible to external devices. For example, controller 150 may be connected to a removable disc via a USB connector or wirelessly communicate with external devices, for example, mobile device 20 (illustrated in FIG. 1A). For example, mobile device 20 may be a smartphone or a tablet associated with a medic providing the initial medical treatment to a patient.

In some embodiments, an application running on mobile device 20 may communicate with controller 150 via communication unit 158 and further may send information stored in memory 156 to be further processed or used by other professionals that may treat the patient, for example, the doctors in the emergency room. In some embodiments, controller 150 may send the information stored on memory 156 to an external device either wirelessly or other connections (e.g., USB plug).

Reference is now made to FIGS. 2A and 2B which are illustrations of plug 200 according to some embodiments of the invention. Plug 200 may include a housing 205 connectable to a contra plug (not illustrated), a first portion 212 configured to connect one or more sterile treatment supplying channels and a second portion 210 configured to connect at least one of: wires and channels 214-228. First portion 212 may be sterile and may be configured to be connected to a sterile channel 112, for example, for supply fluids via IV infusion. In a non-connectable state as illustrated in FIG. 2A the first portion 212 is sealed by a cover 213 having an opening 211. When there is a need to connect first portion 212 to a sterile channel, a sterile connector 212A is pushed out and connects to the fluids channel (e.g., a sterile IV tube) as illustrated in FIG. 2B. In some embodiments, cover 213 may be a tearable membrane configured to protect the one or more sterile channels 212A from external contamination prior to connecting the plug to the contra plug.

Second portion 210 may include a plurality of connectors that are configured to connect sensors and supply channels that do not require a sterile environment. For example, second portion 210 may include a ventilation connector 214 for supplying pressurized air/oxygen to channel such as channel 114. In yet another example, second portion 210 may include connectors 222 for supplying pressurized air for blood pressure measurements, connectors 224 for supplying pressurized air for creating a tourniquet, electrical connectors 225 and 226 to connect thermometer and capnography sensor and the like. Second portion 210 may further include a connector 227 for connecting pulse oximetry device and connector 228 for connecting ECG electrodes. In some embodiments, second portion 210 may also be sterile.

In some embodiments, the external medical condition monitoring unit and the external medical treatment supplying unit may include intervascular (IV) fluid line, IV drug administration line, pneumatic channel, and an air/oxygen supplying channel. In some embodiments, the external medical condition monitoring unit and the external medical treatment supplying unit may be located in an ambulance, accordingly, a patient placed on foldable stretcher 100 and connected to at least some of channels 112-124 may be connected to the units in the ambulance in a single simple act, by plugging plug 200 to the contra plug in the ambulance.

Some embodiments of the invention may be related to a kit for connecting treatment supplying channel and life supporting sensors to an external medical treatment supplying unit and an external medical condition monitoring unit. The kit may include plug 200 and a contra plug (not illustrated). The contra plug may include a housing connectable to the plug, a first portion configured to connect one or more sterile treatment supplying channels, wherein the first portion is sterile and a second portion configured to connect at least one of a wire and a channel. In some embodiments, the plug and the contra plug are configured to be connected and separated in a single act.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A foldable stretcher comprising:
a foldable fabric;
one or more sensors for detecting a medical condition of a patient, attached to the foldable fabric and configured to connect to one or more external medical condition monitoring units; and
one or more treatment supplying channels for supplying medical treatment to the patient, attached to the foldable fabric and configured to connect to one or more treatment supplying units.

2. The foldable stretcher according to claim 1, further comprising a plug configured to connect the one or more sensors and the one or more treatment supplying channels to an external medical condition monitoring unit and an external medical treatment supplying unit in a single plugging act.

3. The foldable stretcher according to claim 2, wherein the plug has a sterile portion for connecting the one or more treatment supplying channels.

4. The foldable stretcher according to claim 1, wherein at least portions of wires connecting the one or more sensors are embedded into the fabric of the fabric stretcher.

5. The foldable stretcher according to claim 1, wherein at least portions of tubes connecting the one or more treatment supplying channels are embedded into the fabric of the fabric stretcher.

6. The foldable stretcher according to claim 1, wherein the one or more treatment supplying channels include at least one of: a fluid line and a pressurized air-line

7. The foldable stretcher according to claim 5, wherein the one or more treatment supplying channels are connected to an Intervascular (IV) needle, IV drug administration needle, a pneumatic cuff for forming tourniquet, and an air/oxygen supplying mask.

8. The foldable stretcher according to claim 1, wherein the one or more sensors include at least one of: heart rate monitoring sensor, an oxygen saturation sensor for measuring oxygen level in the blood (SpO₂), a thermometer and a blood pressure sensor.

9. The foldable stretcher according to claim 1, further comprising:
one or more connectors for connecting the one or more sensors to one or more external medical condition monitoring units.

10. The foldable stretcher according to claim 1, further comprising:
one or more connectors for connecting the one or more treatment supplying channels to one or more treatment supplying units.

11. The foldable stretcher according to claim 1, further comprising:
a controller configured to:
receive information from the one or more sensors; and
store the information in a memory associated with the controller.

12. The foldable stretcher according to claim 11,
wherein the controller further comprises a communication unit, and
wherein the controller is further configured to:
send the information to an external device.

13. A plug for connecting treatment supplying channels and life supporting sensors of a stretcher, to an external medical condition monitoring unit and an external medical treatment supplying unit, comprising:
a housing connectable to a contra plug;
a first portion configured to connect one or more sterile treatment supplying channels, wherein the first portion is sterile; and
a second portion configured to connect at least one of: a wire and a channels.

14. The plug according to claim 13, wherein the first portion comprises a tearable membrane configured to protect the one or more sterile channels from external contamination prior to connecting the plug to the contra plug.

15. The plug according to claim 13, wherein the second portion includes one or more electrical connectors for connecting wires connected to the life supporting sensors.

16. The plug according to claim 13, wherein the treatment supplying equipment include at least one of: Intervascular (IV) fluid line, IV drug administration line, pneumatic channel, and an air/oxygen supplying channel.

17. The plug according to claim 13, wherein the sensors include at least two of: heart rate monitoring sensor, a blood oxygen saturation level sensor, a thermometer and a blood pressure sensor.

18. The plug according to claim 13, wherein the second portion is sterile.

19. A kit for connecting treatment supplying channel and life supporting sensors to an external medical treatment supplying unit and an external medical condition monitoring unit, comprising:
a plug according to any one of claims 13-19; and
a contra plug comprising:
a housing connectable to the plug;
a first portion configured to connect one or more sterile treatment supplying channels, wherein the first portion is sterile; and
a second portion configured to connect at least one of a wire and a channel,
wherein the plug and the contra plug are configured to be connected and separated in a single act.
